(19) United States
(12) Patent Application Publication
(10) Pub. No.: US 2023/0390169 A1
(43) Pub. Date: Dec. 7, 2023

(54) TOPICAL COMPOSITION FOR TREATING DANDRUFF AND SEBORRHEIC DERMATITIS OF A SCALP

(71) Applicants: Alexander Sepper, New York, NY (US); Dennis Tubian, New York, NY (US); John Navi, New York, NY (US)

(72) Inventors: Alexander Sepper, New York, NY (US); Dennis Tubian, New York, NY (US); John Navi, New York, NY (US)

(21) Appl. No.: 18/130,771

(22) Filed: Apr. 4, 2023

Related U.S. Application Data
(63) Continuation of application No. 17/744,701, filed on May 15, 2022, now abandoned.

(51) Int. Cl. A61K 8/27; A61Q 5/00; A61K 8/49; A61K 8/20; A61K 8/46; A61K 8/73

(52) U.S. Cl. CPC A61K 8/27; A61Q 5/006; A61K 8/494; A61K 8/20; A61K 8/466; A61K 8/738; A61K 8/492

(57) ABSTRACT

A topical composition treating dandruff and seborrheic dermatitis of a scalp, by restoring architectonics to the scalp, eliminating fungal infection from the scalp, regulating metabolism of the scalp, regulating production and release of sebum from the scalp, and detoxifying the scalp. The topical composition includes an anti-fungal and scalp metabolism regulator, a delivery system, a detoxifying and impurity eliminator, and a fragrance. The anti-fungal and scalp metabolism regulator includes zinc pyrithione, climbazole, melatonin, multi-functional polyethylene glycol, and hydroxypropyl-ß-cyclodextrin. The delivery system includes first and second solutions. The first solution includes dimethylsulfoxide and hydroxypropyl-ß-cyclodextrin. The second solution includes distilled water, sodium chloride, potassium chloride, calcium chloride, sodium bicarbonate, imidazolidinyl urea, and multi-functional polyethylene glycol. The detoxifying and impurity eliminator includes preservatives, a pathogenic microorganism and exo- and endotoxins eliminator, and multi-functional polyethylene glycol. The preservatives include sodium benzoate and potassium sorbate. The pathogenic microorganism and exo- and endotoxins eliminator includes white activated charcoal powder.

### CROSS REFERENCE TO RELATED APPLICATIONS

The instant non-provisional patent application is a non-provisional Continuation-In-Part patent application of non-provisional patent application Ser. No. 17/744,701 to Sepper et al., filed on 15 May 2022, in group art unit 1629, and which claims priority from U.S. application No. 63/189,444, filed 17 May 2021 and U.S. application No. 63/189,454, filed 17 May 2021. All of which are included in their entity by reference thereto.

### BACKGROUND OF THE INVENTION

### Field of the Invention

The embodiments of the present invention relates to a topical composition for treating dandruff and seborrheic dermatitis of a scalp, and more particularly, the embodiments of the present invention relate to a topical composition for treating dandruff and seborrheic dermatitis of a scalp, by restoring architectonics to the scalp, by eliminating fungal infection from the scalp, by regulating metabolism of the scalp, by regulating production and release of sebum from the scalp, and by detoxifying the scalp.

### Description of the Prior Art

Conditioners

Conditioning of hair is often essential to good grooming. This is particularly the case if the hair is of fine texture or excessively dry. In other instances, conditioning is used when hair has become brittle or straw-like, as a result of, excessive use of hair dyes, bleaches, blow-dryers, and the like.

With the advent of blow-dryers for drying hair, excessive exposure to heat can leave the hair in a limp and tortured state. Conditioners in common, use protein enriched lotions, creams, and numerous oil-based materials that are applied to the hair followed by rinsing to remove excess conditioner. The degree with which hair absorbs the conditioner varies both with the conditioner used and the state of the hair. Most conditioners have a tendency to leave the hair with an oily-greasy feel that can be undesirable. The primary reason for this is that the amount of conditioner applied is in excess of the amount absorbed by each hair strand.

Falling Hair

Furthermore, disorders of the scalp can lead to a widespread problem known as “falling hair.”Causes of this problem are not yet fully understood. While it is believed that heredity plays a major role, it is generally considered that this factor is not the only one at work. Other factors, such as, stress, poor nutrition, and excessive oiliness of the scalp are also believed to be involved in the cause and effect of this problem.

Defective Keratinization

Disease conditions characterized by defects in keratinization are also relatively common, and many different treatments have been prescribed in the past with varying degrees of effectiveness. In each of these disease conditions, the process whereby the epidermal cells mature and form a surface layer—called stratum corneum—is defective. Therefore, the signs and symptoms of diseases associated with defective keratinization are an overproduction of cells and or their retention in the stratum corneum for abnormally prolonged periods.

Seborrhea Capitis

Disorders of the scalp can also include a condition known as seborrhea capitis. This involvement is characterized by thick and yellowish greasy scales of the vertex and possibly covering most of the scalp. In many cases the hair may be matted with crusts that may vary in color. They frequently have a dirty and waxy appearance, and present a most distressing cosmetic problem. When the scaling is dry, it is given the medical diagnosis of pityriasis sicca, and when the scaling is oily, it is given the medical diagnosis of pityriasis oleos. Since there is no known cure for seborrhea type disorders, treatment must by necessity be aimed at the most effective method that will achieve symptomatic relief. Effective ways of control should include a way for cleaning and clearing the scalp scales and oiliness.

Dandruff

A hair and scalp problem of widespread importance is the itching, scaling, and sloughing of epidermal cells of the scalp, referred to as common dandruff. Dandruff is known to be a complex condition. It is believed that a dandruff condition involves several factors, such as, the presence of staphylococcus aureus and pittosporum ovule skin bacteria, along with oily, dead, and surface epidermal cells. Growth of these microbes is believed to promote or at least to be associated with the itching, scaling, and unpleasant appearance associated with increased desquamation of epidermal cells.

Anti-Dandruff Shampoos

Anti-dandruff shampoos have been prevailing on the market for many years. Additionally, according to epidemiological data, the frequency of dandruff and seborrheic dermatitis problems is increasing, apparently as a result of the constantly growing level of industrialization of many aspects of our life, including nutrition and the market for various personal and household hygiene products, which obviously contribute to the development of metabolic and hormonal abnormalities in cellular activity and function of the cellular elements of the scalp and hair follicles. Anti-dandruff compositions have involved use of germicides in detergent bases many of which can unfortunately be very harsh to the skin, hair, and eyes.

Vitamin A

Vitamin A has a well-established chemical structure. It is commercially available from a variety of sources.¹ Vitamin A is considered to be insoluble in water. There is a widespread use of vitamin A in ointment bases, cosmetics, and topical pharmaceutical preparations, however, all vitamin A containing aqueous compositions are guilty of cloudy emulsions and grease like masses. Application of these type materials to the hair and scalp have enjoyed limited use, as a result of, the greasy and often messy after-effect of their use. ¹One source is Roche Chemical Company of Nutley, New Jersey.

Sorbed Materials

In literature publications², it was demonstrated that amino acid sorption onto the hair occurs in a very uneven manner. Wide variation is noted along the hair shaft, as determined at intervals outward from the root end. In order for effective conditioning of hair and improvement in manageability, even distribution of sorbed materials is desirable. ²The Journal of the Society of Cosmetic Chemists of Great Britain, Vol. 10, Number 4, (1959) under an article entitled “The Sorption of Amino Acids from Shampoo on to Hair”, by Herd, J. K., et al,

Additional Prior Art

Numerous means for skin treatments have been provided in the prior art, which will be described, infra, in chronological order to show possible advancement in the art, and which are incorporated herein in their entirety by reference thereto. Even though these means may be suitable for the specific individual purposes to which they address nevertheless they differ from the embodiments of the present invention in that they do not explicitly disclose a topical composition for treating dandruff and seborrheic dermatitis of a scalp, by restoring architectonics to the scalp, by eliminating fungal infection from the scalp, by regulating metabolism of the scalp, by regulating production and release of sebum from the scalp, and by detoxifying the scalp. For example:

U.S. Pat. No. 3,920,835A to Van Scott et al.

U.S. Pat. No. 3,920,835A issued to Van Scott et al. on 18 Nov. 1975 in U.S. class 424 and subclass 311 teaches a treatment to alleviate the symptoms of diseases characterized by defects in keratinization. The treatment includes the topical application of an ointment or lotion containing one or more lower aliphatic compounds having from two to about six carbon atoms, and preferably, having α-carbon functionality. The compounds include alpha-hydroxy acids, keto acids and esters thereof, and 3-hydroxybutyric acid. The therapeutic composition may include one or more of the compounds present in a total of from 1-20% —either a water solution or an alcohol solution or an ointment.

U.S. Pat. No. 3,984,566A to Van Scott et al.

U.S. Pat. No. 3,984,566A issued to Van Scott et al. on 5 Oct. 1976 in U.S. class 424 and subclass 283 teaches a treatment to alleviate the symptoms of diseases characterized by defects in keratinization. The treatment includes the topical application of an ointment or lotion containing one or more lower aliphatic compounds having from 2 to about 6 carbon atoms, and preferably, having α-carbon functionality. The compounds include α-hydroxy acids, keto acids and esters thereof, and 3-hydroxybutyric acid. The therapeutic composition may include one or more of the compounds present in a total amount of from 1-20% percent in either a water solution Of an alcohol solution or an ointment.

U.S. Pat. No. 4,105,782 to Yu et al.

U.S. Pat. No. 4,105,782 issued to Yu et al. on 8 Aug. 1978 in U.S. class 424 and subclass 283 teaches a preventive, as well as, a therapeutic treatment to alleviate the symptoms of diseases characterized by defects in keratinization. The therapeutic treatment includes the topical application of a solution, a lotion, a cream, or a shampoo containing one of more of the α-hydroxy or α-keto acid amides and of ammonium salts. The compounds include, amide and or ammonium salt forms of citric acid, glycolic acid, glucuronic acid, galacturonic acid, glucuronolactone, gluconolactone, α-hydroxybutyric acid, α-hydroxyisobutyric acid, magic acid, mandate acid, pyruvic acid, β-phenylacetic acid, β-phenyl pyruvic acid, saccharic acid, tartaric acid, tartronic acid, and β-hydroxybutyric acid. The therapeutic composition may include one or more of the compounds present in a total amount of from 1-20%.

U.S. Pat. No. 4,170,229 to Olson

U.S. Pat. No. 4,170,229 issued to Olson on 9 Oct. 1979 in U.S. class 128 and subclass 67 teaches vitamin A-containing, crystal-clear, transparent, aqueous, and sprayable emulsions for application to hair and scalp, according to a spray-on then brash-in technique.

U.S. Pat. No. 5,221,533 to Pearlman

U.S. Pat. No. 5,221,533 issued to Pearlman on 22 Jun. 1993 in U.S. class 424 and subclass 73 teaches a skin lotion and methods of use involving a skin lotion including effective amounts of allantoin, castor oil, oleic acid, calcium hydroxide powder, and witch hazel water. The methods of use include as a keratoplasty and keratolytic for removing the piled up hyperkeratotic scales in seborrheic capitis and psoriasis vulgaris to expedite the involution of the lesions of acute herpes simplex.

U.S. Pat. No. 6,048,886A to Neigut

U.S. Pat. No. 6,048,886A issued to Neigut on 11 Apr. 2000 in U.S. class 514 and subclass 412 teaches compositions and methods for application of those compositions in order to treat biological surfaces, especially the skin. The compositions contain a ubiquinone, may include other antioxidant agents, including melatonin and may include an abrasive. The way of application may be by rubbing the composition into the skin, by way of a transdermal patch, or by way of rubbing the composition into the skin followed by application of a transdermal patch.

U.S. Pat. No. 6,168,798 to O'Halloran et al.

U.S. Pat. No. 6,168,798 issued to O'Halloran et al. on 2 Jan. 2001 in U.S. class 424 and subclass 401 teaches methods and compositions for reducing and eliminating skin disorders, particularly, blemishes and redness associated with acne. The hydroalcoholic compositions contain a keratolytic compound, preferably, a β-hydroxy carboxylic acid, more preferably, salicylic acid or pharmaceutically acceptable salts thereof and a glycerophosphate ester or salt formulated with pharmaceutically acceptable carriers. Glycerophosphate salts for use in the methods and compositions are sodiumglycerophosphate and calcium glycerophosphate. Other components that may be formulated into the compositions include fragrances, colorants, emollients, humectants, and antimicrobial agents.

Korean Publication Number KR 19990026954A Kang et al.

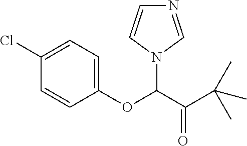 [SMILES: CC(C)(C)C(=O)C(Oc1ccc(Cl)cc1)n1ccnc1]

Korean publication number KR19990026954A published to Kang et al. on February 2001 teaches an anti-dandruff shampoo composition containing climbazole and zinc pyrithione.

WIPO Publication Number WO 20071099398 A2 to DeRijk

WIPO publication number WO 2007/099398 A2 published to DeRijk on 7 Sep. 2007 in CPC classification A01N37/36 teaches compositions and their use for the treatment of human skin, particularly, facial skin to alleviate the symptoms of cosmetic or dermatologic skin conditions, and for treating and ameliorating skin conditions including acne, rosacea, and wrinkling caused by photodamage or conditions related to aging, hormonal imbalances, hyper-pigmentation, melasma, keratosis, or the like, and to treat conditions of the scalp, such as, dandruff. The compositions can be used to remove biofilms from contact lenses. More particularly, the composition includes at least one metasilicates, at least one carbonates, at least one glycinates, and at least one sulfates. The composition may also contain salts, e.g., sea salts and other additives or active agents.

WIPO Publication Number WO 2016/123223A1 to Abreo et al.

WIPO publication number WO 2016/123223 A1 published to Abreo et al. on 4 Aug. 2016 in CPC class A61F13/00063 teaches stabilized compositions for treating skin conditions, wherein a therapeutically effective dosage of a sulfur compound is applied. The formulations and the methods are applicable to a variety of skin conditions including cold sores, herpes, genital herpes, and shingles.

EMA, Cycoldextrins Used as Excipients, 2017, pages 1-16 (Year: 2017)

A report published in support of the questions and answers on cycoldextrins used as excipients in medical products in human use.

Drewitt Princeton Consumer Research Global Product

Testing (Year 2019) (Henceforth Referred to as “DREWITT”)³ ³https//essentiaremedium.com/products/dandr-o-heal/.

DREWITT discloses a double-blind study to access the efficacy of a dandruff product via clinical grading, clinical assessment, and self-perception questionaries.

Applicants respectfully submit herewith a declaration of Applicant Dennis Tubian disqualifying DREWITT as prior art, since as the declaration will prove, DREWITT was made public less than one year before the oldest priority date of the instant CIP application.

### The Distinctive Advantageous Results of the Embodiments of the Present Invention

It is apparent that numerous means for skin treatments have been provided in the prior art, which are adapted to be used. Furthermore, even though these means may be suitable for the specific individual purposes to which they address, nevertheless, they would not be suitable for the purposes of the embodiments of the present invention, as heretofore described, because in contradistinction, none of the references cited, supra, appear to explicitly disclose Applicants' following advantageous results⁴: ⁴Ex parte Tanaka, Marushima and Takahashi, 174 USPQ 38 (“Claims are not rejected . . . if the prior art devices do not accomplish applicant's result.”[Emphasis added] and In re Dillon, 919 F.2d 688, 692 (Fed. Cir. 1990) (“A prima facie case of . . . [unpatentability] requires that the prior art suggest the . . . problem the applicant attempts to solve.”[Emphasis added].
Restores architectonics to the scalp;
Eliminates fungal infection from the scalp;
Regulates metabolism of the scalp;
Regulates production of sebum from the scalp;
Regulates release of sebum from the scalp;
Detoxifies the scalp;
Stabilizes the structure of substances integrated into it, and thus, prolongs their
activity in a specific way, in delivering the target ingredients to target tissues;
Possesses fungistatic and bacteriostatic properties;
Possesses antifungal properties;
Synchronizes circadian rhythms of the scalp;
Eliminates pathogenic microorganism and exo- and endotoxins from the scalp;
Binds with harmful fungi and fungal debris and byproducts of the scalp;
Removes harmful fungi and fungal debris and byproducts from the scalp;
Reduces toxicity of the scalp;
Strengthens immune defense of the scalp;
Works immediately after application to the scalp, so there is no waiting period;
Protects the scalp from harsh environmental influences;
Does not irritate the scalp;
Eliminates completely from the scalp by water; and
Does not interfere with hair colorants used on the scalp.

### SUMMARY OF THE INVENTION

Thus, an object of the embodiments of the present invention is to provide a topical composition for treating dandruff and seborrheic dermatitis of a scalp, by restoring architectonics to the scalp, by eliminating fungal infection of the scalp, by regulating metabolism of the scalp, by regulating production and release of sebum from the scalp, and by detoxifying the scalp which avoids the disadvantages of the prior art.

Briefly stated, another object of the embodiments of the present invention is to provide a topical composition that treats dandruff and seborrheic dermatitis of a scalp, by restoring architectonics to the scalp, by eliminating fungal infection from the scalp, by regulating metabolism of the scalp, by regulating production and release of sebum from the scalp, and by detoxifying the scalp. The topical composition includes an anti-fungal and scalp metabolism regulator, a delivery system, a detoxifying and impurity eliminator, and a fragrance. The anti-fungal and scalp metabolism regulator includes zinc pyrithione, climbazole, melatonin, multi-functional polyethylene glycol, and hydroxypropyl-ß-cyclodextrin. The delivery system includes a first solution and a second solution. The first solution of the delivery system includes dimethylsulfoxide (DMSO) and hydroxypropyl-ß-cyclodextrin. The second solution of the delivery system includes distilled water, sodium chloride, potassium chloride, calcium chloride, sodium bicarbonate, imidazolidinyl urea, and multi-functional polyethylene glycol. The detoxifying and impurity eliminator includes preservatives, a pathogenic microorganism and exo- and endotoxins eliminator, and multi-functional polyethylene glycol. The preservatives of the detoxifying and impurity eliminator include sodium benzoate and potassium sorbate. The pathogenic microorganism and exo- and endotoxins eliminator of the detoxifying and impurity eliminator includes white activated charcoal powder.

Still another object of the embodiments of the present invention is to provide a topical composition for treating dandruff and seborrheic dermatitis of a scalp, by restoring architectonics to the scalp, by eliminating fungal infection from the scalp, by regulating metabolism of the scalp, by regulating production and release of sebum from the scalp, and by detoxifying the scalp, and which includes an anti-fungal and scalp metabolism regulator, a delivery system, and a detoxifying and impurity eliminator. The detoxifying and impurity eliminator includes a pathogenic microorganism and exo- and endotoxins eliminator. The pathogenic microorganism and exo- and endotoxins eliminator of the detoxifying and impurity eliminator includes white activated charcoal powder.

Yet another object of the embodiments of the present invention is to provide a topical composition whose anti-fungal and scalp metabolism regulator includes zinc pyrithione having fungistatic and bacteriostatic properties, climbazole being a topical antifungal agent, melatonin being a hormone involved in synchronizing circadian rhythms, multi-functional polyethylene glycol, and hydroxypropyl-ß-cyclodextrin.

Still yet another object of the embodiments of the present invention is to provide a topical composition whose zinc pyrithione of the anti-fungal and scalp metabolism regulator is in a concentration range of 0.05-5.7% wt/wt, whose climbazole of the anti-fungal and scalp metabolism regulator is in a concentration range of 1.0-6.0% wt/wt, and whose melatonin of the anti-fungal and scalp metabolism regulator is in a concentration range of 0.03-3.7% wt/wt.

Yet still yet another object of the embodiments of the present invention is to provide a topical composition whose zinc pyrithione of the anti-fungal and scalp metabolism regulator is in a concentration range of 0.01-10% wt/wt, whose climbazole of the anti-fungal and scalp metabolism regulator is in a concentration range of 0.5-10% wt/wt, and whose melatonin of the anti-fungal and scalp metabolism regulator is in a concentration range of 0.01-5% wt/wt.

Still yet another object of the embodiments of the present invention is to provide a topical composition whose zinc pyrithione of the anti-fungal and scalp metabolism regulator is in a concentration range of 0.05-10% wt/wt, whose climbazole of the anti-fungal and scalp metabolism regulator is in a concentration range of 0.75-10% wt/wt, and whose melatonin of the anti-fungal and scalp metabolism regulator is in a concentration range of 0.02-4% wt/wt.

Yet still another object of the embodiments of the present invention is to provide a topical composition whose zinc pyrithione of the anti-fungal and scalp metabolism regulator is in a concentration range of 0.1-10% wt/wt, and whose climbazole of the anti-fungal and scalp metabolism regulator is in a concentration range of 1.0-6% wt/wt.

Still yet another object of the embodiments of the present invention is to provide a topical composition whose zinc pyrithione of the anti-fungal and scalp metabolism regulator is in a concentration range of 0.2-10% wt/wt.

Yet still another object of the embodiments of the present invention is to provide a topical composition whose delivery system includes a first solution and a second solution.

Still yet another object of the embodiments of the present invention is to provide a topical composition whose first solution of the delivery system includes dimethylsulfoxide (DMSO) and hydroxypropyl-ß-cyclodextrin (HPBC).

Yet still another object of the embodiments of the present invention is to provide a topical composition whose dimethylsulfoxide (DMSO) of the first solution of the delivery system is in a concentration range of 4.0-36.0% wt/wt, and whose hydroxypropyl-ß-cyclodextrin (HPBC) of the first solution of the delivery system is in a concentration range of 0-75-27.0% wt/wt.

Still yet another object of the embodiments of the present invention is to provide a topical composition whose second solution of the delivery system includes distilled water, sodium chloride, potassium chloride, calcium chloride, sodium bicarbonate, imidazolidinyl urea, and multi-functional polyethylene glycol.

Yet still another object of the embodiments of the present invention is to provide a topical composition whose distilled water of the second solution of the delivery system is in a concentration range of 8.0-46.0% wt/wt, whose potassium chloride of the second solution of the delivery system is in a concentration range of 0.7-2.8% wt/wt, whose calcium chloride of the second solution of the delivery system is in a concentration range of 0.03-5.2% wt/wt, whose sodium bicarbonate of the second solution of the delivery system is in a concentration range of 0.02-6.1% wt/wt, and whose imidazolidinyl urea of the second solution of the delivery system is in a concentration range of 0.1-4.5% wt/wt.

Still yet another object of the embodiments of the present invention is to provide a topical composition whose distilled water of the second solution of the delivery system is in a concentration range of 8-90% wt/wt,whose potassium chloride of the second solution of the delivery system is in a concentration range of 0.01-5% wt/wt, whose calcium chloride of the second solution of the delivery system is in a concentration range of 0.01-5% wt/wt, whose sodium bicarbonate of the second solution of the delivery system is in a concentration range of 0.01-10% wt/wt, and whose imidazolidinyl urea of the second solution of the delivery system is in a concentration range of 0.01-10% wt/wt.

Yet still another object of the embodiments of the present invention is to provide a topical composition whose detoxifying and impurity eliminator includes preservatives, a pathogenic microorganism and exo- and endotoxins eliminator, and multi-functional polyethylene glycol.

Still yet another object of the embodiments of the present invention is to provide a topical composition whose preservatives of the detoxifying and impurity eliminator include sodium benzoate and potassium sorbate.

Yet still another object of the embodiments of the present invention is to provide a topical composition whose sodium benzoate of the preservatives of the detoxifying and impurity eliminator is in a concentration of 0.500% wt/wt, and whose potassium sorbate of the preservatives of the detoxifying and impurity eliminator is in a concentration of 0.050% wt/wt.

Still yet another object of the embodiments of the present invention is to provide a topical composition whose white activated charcoal powder of the pathogenic microorganism and exo- and endotoxins eliminator of the detoxifying and impurity eliminator is 10% of the topical composition, and which includes colloidal silicon dioxide that is submicroscopic fumed silica prepared by vapor-phase hydrolysis of a silicon compound.

Yet still another object of the embodiments of the present invention is to provide a topical composition whose white activated charcoal powder of the pathogenic microorganism and exo- and endotoxins eliminator of the detoxifying and impurity eliminator binds harmful fungi and fungal debris and byproducts of the scalp, removes harmful fungi and fungal debris and byproducts from the scalp, regulates and reduces toxicity and detoxifies the scalp, strengthens immune defense of the scalp, starts to work immediately after application to the scalp, protects the scalp from harsh environmental influences, does not irritate the scalp, is completely eliminated from the scalp by water, and does not interfere with hair colorants.

Still yet another object of the embodiments of the present invention is to provide a topical composition that restores architectonics to the scalp, eliminates fungal infection from the scalp, regulates metabolism of the scalp, regulates production and release of sebum from the scalp, detoxifies the scalp, stabilizes structure of substances integrated into it, and thus, prolongs their activity in a specific way in delivering target ingredients to target tissues, provides fungistatic and bacteriostatic properties to the scalp, provides antifungal properties, synchronizes circadian rhythms of the scalp, binds to harmful fungi and fungal debris and byproducts of the scalp, removes harmful fungi and fungal debris and byproducts from the scalp, regulates toxicity and detoxifies the scalp, reduces toxicity and detoxifies the scalp, strengthens immune defense of the scalp, starts to work immediately after application to the scalp, protects the scalp from harsh environmental influences, does not irritate the scalp, is completely eliminated from the scalp by water, and does not interfere with hair colorants on the scalp.

Yet still another object of the embodiments of the present invention is to provide a topical composition whose anti-fungal and scalp metabolism regulator stabilizes structure of substances integrated into it, and thus, prolongs their activity in a specific way in delivering target ingredients to target tissues, whose zinc pyrithione of the anti-fungal and scalp metabolism regulator provides fungistatic and bacteriostatic properties to the scalp, whose climbazole of the anti-fungal and scalp metabolism regulator provides antifungal properties, whose melatonin of the anti-fungal and scalp metabolism regulator synchronizes circadian rhythms of the scalp, whose white activated charcoal powder of the pathogenic microorganism and exo- and endotoxins eliminator of the detoxifying and impurity eliminator eliminates pathogenic microorganism and exo- and endotoxins from the scalp, binds to harmful fungi and fungal debris and byproducts of the scalp, removes harmful fungi and fungal debris and byproducts from the scalp, regulates toxicity and detoxifies the scalp, reduces toxicity of the scalp, strengthens immune defense of the scalp, starts to work immediately after application to the scalp, protects the scalp from harsh environmental influences, does not irritate the scalp, is completely eliminated from the scalp by water, and does not interfere with hair colorants on the scalp.

Still yet another object of the embodiments of the present invention is to provide a topical composition that further includes a fragrance.

The novel features considered characteristic of the embodiments of the present invention are set forth in the appended claims. The embodiments of the present invention themselves, however, both as to their construction and to their method of operation—together with additional objects and advantages thereof will be best understood from the following description of the embodiments of the present invention when read and understood in connection with the accompanying figures of the drawings.

### BRIEF DESCRIPTION OF THE FIGURES OF THE DRAWINGS

FIG. 1 is a block diagram of the configuration of the topical composition of the embodiments of the present invention;

FIG. 2 is a block diagram of the configuration of the anti-fungal and scalp metabolism regulator of the topical composition of the embodiments of the present invention;

FIG. 3 is a block diagram of the configuration of the delivery system of the topical composition of the embodiments of the present invention;

FIG. 4 is a block diagram of the configuration of the first solution of the delivery system of the topical composition of the embodiments of the present invention;

FIG. 5 is a block diagram of the configuration of the second solution of the delivery system of the topical composition of the embodiments of the present invention;

FIG. 6 is a block diagram of the configuration of the detoxifying and impurity eliminator of the topical composition of the embodiments of the present invention;

FIG. 7 is a block diagram of the configuration of the preservatives of the detoxifying and impurity eliminator of the topical composition of the embodiments of the present invention; and

FIG. 8 is a block diagram of the configuration of the pathogenic microorganism and exo- and endotoxins eliminator of the detoxifying and impurity eliminator of the topical composition of the embodiments of the present invention.

### LIST OF REFERENCE NUMERALS UTILIZED IN THE FIGURES OF THE DRAWINGS

### Introductory

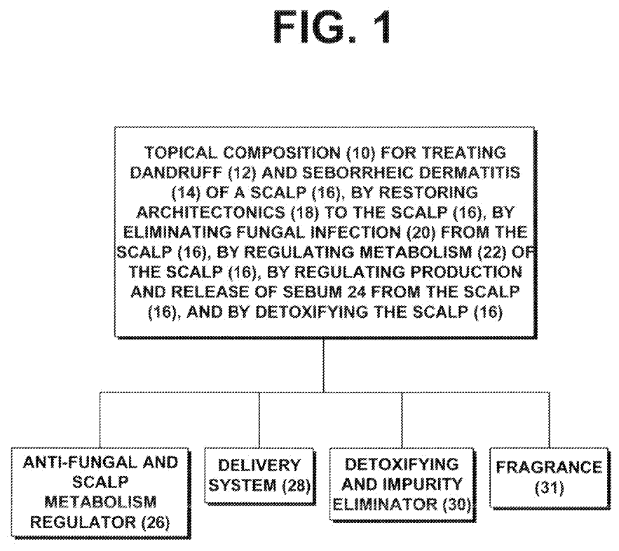

(FIG. 1)
10 topical composition of embodiments of present invention for treating dandruff 12 and seborrheic dermatitis 14 of scalp 16, by restoring architectonics 18 to scalp 16, by eliminating fungal infection 20 from scalp 16, by regulating metabolism 22 of scalp 16, by regulating production and release of sebum 24 from scalp 16, and by detoxifying scalp 16
12 dandruff of scalp 16
14 seborrheic dermatitis of scalp 16
16 scalp
18 architectonics of scalp 16
20 fungal infection of scalp 16
22 metabolism of scalp 16
24 sebum of scalp 16

### Overall Configuration of Composition 10

(FIG. 1)
26 anti-fungal and scalp metabolism regulator
28 delivery system
30 detoxifying and impurity eliminator
31 fragrance

### Specific Configuration of Anti-Fungal and Scalp Metabolism Regulator 26

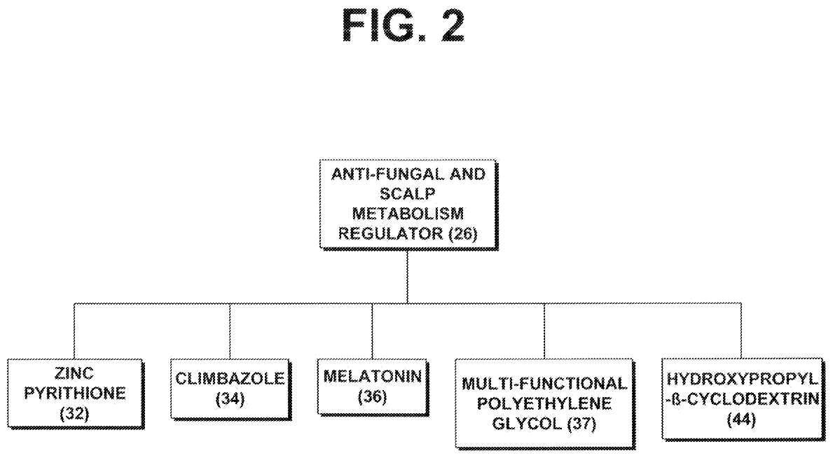

(FIG. 2)
32 zinc pyrithione
34 climbazole
36 melatonin
37 multi-functional polyethylene glycol

### Specific Configuration of Delivery System 28

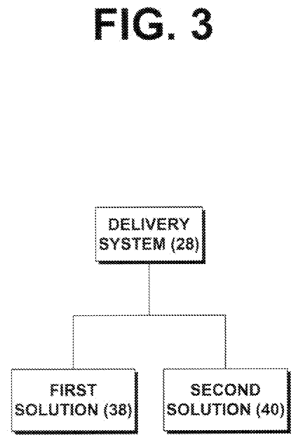

(FIG. 3)
38 first solution
40 second solution

### Specific Configuration of First Solution 38 of Delivery System 28

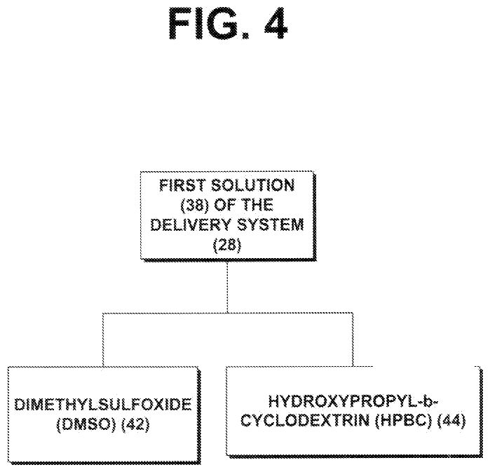

(FIG. 4)
42 dimethylsulfoxide (DMSO)
44 hydroxypropyl-β-cyclodextrin (HPBC)

### Specific Configuration of Second Solution 34 of Delivery System 28

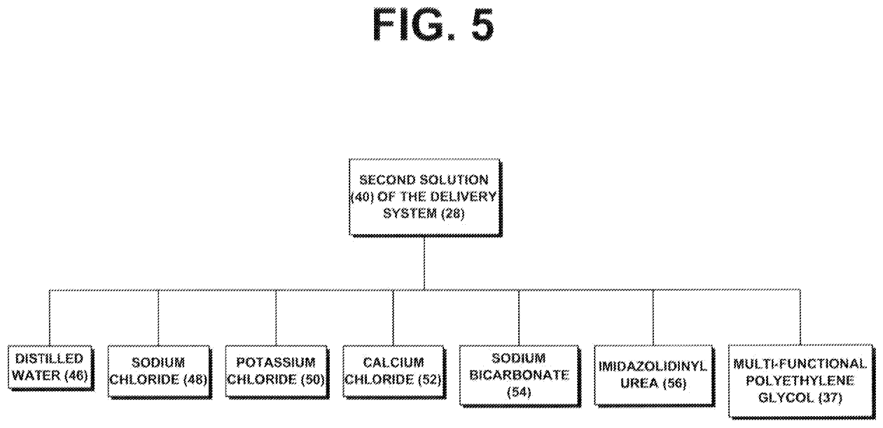

(FIG. 5)
46 distilled water
48 sodium chloride
50 potassium chloride
52 calcium chloride
54 sodium bicarbonate
56 imidazolidinyl urea

### Specific Configuration of Detoxifying and Impurity Eliminator 30

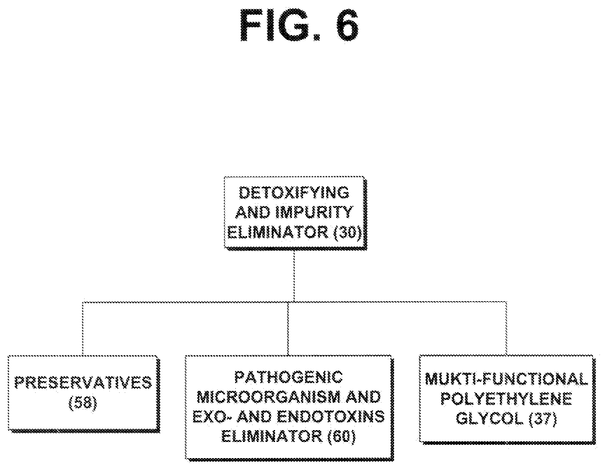

(FIG. 6)
58 preservatives
60 pathogenic microorganism and exo- and endotoxins eliminator

### Specific Configuration of Preservatives 58 of Detoxifying and Impurity Eliminator 30

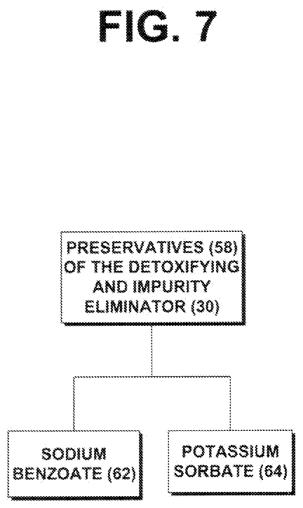

(FIG. 7)
62 sodium benzoate
64 potassiumsorbate

### Specific Configuration of Pathogenic Microorganism and Exo- and Endotoxins Eliminator 60 of Detoxifying and Impurity Eliminator 30

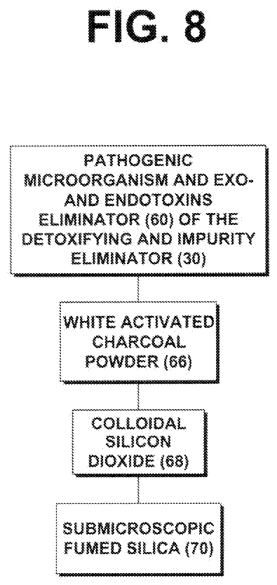

(FIG. 8)
66 white activated charcoal powder
68 colloidal silicon dioxide of white activated charcoal powder 66
70 submicroscopic fumed silica of colloidal silicon dioxide 68 of white activated charcoal powder 66

### DETAILED DESCRIPTION OF THE PREFERRED EMBODIMENTS

### Introductory

Referring now to the figures of the drawing, in which like numerals indicate like parts, and particularly to FIG. 1, the topical composition of the embodiments of the present invention is shown generally at 10 for treating dandruff 12 and seborrheic dermatitis 14 of a scalp 16, by restoring architectonics 18 to the scalp 16, by eliminating fungal infection 20 from the scalp 16, by regulating metabolism 22 of the scalp 16, by regulating production and release of sebum 24 from the scalp 16, and by detoxifying the scalp 16.

### Overall Configuration of the Composition 10

The overall configuration of the topical composition 10 can best be seen in FIG. 1, and as such, will be discussed with reference thereto.

The topical composition 10 includes:
An anti-fungal and scalp metabolism regulator 26;
A delivery system 28;
A detoxifying and impurity eliminator 30; and
A fragrance 31.

### Specific Configuration of the Anti-Fungal and Scalp Metabolism Regulator 26

The specific configuration of the anti-fungal and scalp metabolism regulator 26 can best be seen in FIG. 2, and as such, will be discussed with reference thereto.

The anti-fungal and scalp metabolism regulator 26 stabilizes structure of substances integrated into it, and thus, prolongs their activity in a specific way, and delivers target ingredients to target tissues.

The anti-fungal and scalp metabolism regulator 26 includes:
Zinc pyrithione 32 having fungistatic and bacteriostatic properties, is in a preferred concentration range of 0.05-5.7% wt/wt, and is in alternate concentration ranges of 0.05-10% wt/wt, 0.1-10% wt/wt, and 0.2-10 % wt/wt;
Climbazole 34 is a topical antifungal agent, is in a preferred concentration range of 1.0-6.0% wt/wt, and is in alternate concentration ranges of 0.5-10% wt/wt, and
Melatonin 36 is a hormone involved in synchronizing circadian rhythms, is in a preferred concentration range of 0.03-3.7% wt/wt, and is in alternate concentration ranges of 0.01-5% wt/wt and 0.02-4% wt/wt;
Multi-functional polyethylene glycol 37 is a scalp metabolism regulator; and
Hydroxypropyl-ß-cyclodextrin 44 is a scalp metabolism regulator.

### Specific Configuration of the Delivery System 28

The specific configuration of the delivery system 28 can best be seen in FIG. 3, and as such, will be discussed with reference thereto.

The delivery system 28 includes:
A first solution 38; and
A second solution 40.

### Specific Configuration of the First Solution 38 of the Delivery System 28

The specific configuration of the first solution 38 of the delivery system 28 can best be seen in FIG. 4, and as such, will be discussed with reference thereto.

The first solution 38 of the delivery system 28 includes:
Dimethylsulfoxide (DMSO) 42 in a preferred concentration range of 4.0-36.0% wt/wt; and
Hydroxypropyl-ß-cyclodextrin (HPBC) 44 in a preferred concentration range of 0.75-27.0% wt/wt.

### Specific Configuration of the Second Solution 40 of the Delivery System 28

The specific configuration of the second solution 40 of the delivery system 28 can best be seen in FIG. 5, and as such, will be discussed with reference thereto.

The second solution 40 of the delivery system 28 includes:
Distilled water 46 in a preferred concentration range of 8.0-46.0% wt/wt, and in alternate concentration ranges of 8-90% wt/wt, 10-80% wt/wt, 10-70% wt/wt, 10-50% wt/wt, 10-30% wt/wt, and in 10-20% wt/wt;
Sodium chloride 48 in a preferred concentration range of 0.2-4.3% wt/wt, and in alternate concentration ranges of 0.01-5% wt/wt, 0.5-3% wt/wt, and 1-2% wt/wt;
Potassium chloride 50 in a preferred concentration range of 0.7-2.8% wt/wt, and in alternate concentration ranges of 0.01-5% wt/wt, 0.5-3% wt/wt, and 1-2% wt/wt;
Calcium chloride 52 in a preferred concentration range of 0.03-5.2% wt/wt, and in alternate concentration ranges of 0.01-5% wt/wt, 0.5-3% wt/wt, and 1-2% wt/wt;
Sodium bicarbonate 54 in a preferred concentration range of 0.02-6.1% wt/wt, and in alternate concentration ranges of 0.01-10% wt/wt, 0.1-7% wt/wt, 0.02-6.1% wt/wt, and 0.1-4.5% wt/wt;
Imidazolidinyl urea 56 in a preferred concentration range of 0.1-4.5% wt/wt, and in alternate concentration ranges of 0.01-10% wt/wt, 0.1-7% wt/wt, 0.2-6% wt/wt, and 0.1-4.5% wt/wt; and
Multi-functional polyethylene glycol 37.

### Specific Configuration of the Detoxifying and Impurity Eliminator 30

The specific configuration of the detoxifying and impurity eliminator 30 can best be seen in FIG. 6, and as such, will be discussed with reference thereto.

The detoxifying and impurity eliminator 30 includes:
Preservatives 58;
A pathogenic microorganism and exo- and endotoxins eliminator 60; and
Multi-functional polyethylene glycol 37.

### Specific Configuration of the Preservatives 58 of the Detoxifying and Impurity Eliminator 30

The specific configuration of the preservatives 58 of the detoxifying and impurity eliminator 30 can best be seen in FIG. 7, and as such, will be discussed with reference thereto.

The preservatives 58 of the detoxifying and impurity eliminator 30 include:
Sodium benzoate 62 in a preferred concentration of 0.500% wt/wt; and
Potassium sorbate 64 in a preferred concentration of 0.050% wt/wt.

### Specific Configuration of the Pathogenic Microorganism and Exo- and Endotoxins Eliminator 60 of the Detoxifying and Impurity Eliminator 30

The specific configuration of the pathogenic microorganism and exo- and endotoxins eliminator 60 of the detoxifying and impurity eliminator 30 can best be seen in FIG. 8, and as such, will be discussed with reference thereto.

The pathogenic microorganism and exo- and endotoxins eliminator 60 of the detoxifying and impurity eliminator 30 includes white activated charcoal powder 66.

The white activated charcoal powder 66 of the pathogenic microorganism and exo- and endotoxins eliminator 60 of the detoxifying and impurity eliminator 30 is sold under the trade name CARBOWHITE®, is 10% of the topical composition 10, and includes colloidal silicon dioxide 68, which is submicroscopic fumed silica 70 prepared by vapor-phase hydrolysis of a silicon compound.

The white activated charcoal powder 66 of the pathogenic microorganism and exo- and endotoxins eliminator 60 of the detoxifying and impurity eliminator 30:
Binds and removes harmful fungi and fungal debris and byproducts from the scalp 16;
Regulates and reduces possible toxicity and detoxifies the scalp 16;
Strengthens immune defense of the scalp 16;
Starts to work immediately after application to the scalp 16;
Protects the scalp 16 from harsh environmental influences;
Does not irritate the scalp 16;
Is completely eliminated from the scalp 16 by water; and
Does not interfere with hair colorants.

### Advantageous Results

It has been held that a prior art reference must accomplish applicant's result⁵, and that there is nothing wrong in claiming something by what it does, as opposed to what it is⁶. ⁵Ex parte Tanaka, Marushima and Takahashi, 174 USPQ 38 (“Claims are not rejected . . . if the prior art devices do not accomplish applicant's result.”[Emphasis added] and In re Dillon, 919 F.2d 688, 692 (Fed. Cir. 1990) (“A prima facie case of . . . [unpatentability] requires that the prior art suggest the . . . problem the applicant attempts to solve.”[Emphasis added].⁶In re Swinehart and Sfiligoj, 169 USPQ 226,228 (CCPA 1971) [“We take the characterization “functional,” as used By the Patent Office and argued by the parties, to indicate nothing more than the fact that an attempt is being made to define something (in this case, a composition) by what it does rather than by what it is (as evidenced by specific structure or material, for example). In our view, there is nothing intrinsically wrong with the use of such a technique in drafting patent claims. Indeed we have even recognized in the past the practical necessity for the use of functional language [Emphasis added].

The topical composition 10 accomplishes the following advantageous results, inter alia, because the topical composition 10 is a biologically active and multi-functional complex:
Restores architectonics to the scalp 16, which is accomplished by the hydroxypropyl-ß-cyclodextrin 44 of the first solution 38 of the delivery system 28, the melatonin 36 of the anti-fungal and scalp metabolism regulator 26, and the multi-functional polyethylene glycol 37;
Eliminates fungal infection from the scalp 16, which is accomplished by the zincpyrythione 32 of the anti-fungal and scalp metabolism regulator 26, the climbazole 34 of the anti-fungal and scalp metabolism regulator 26, and the potassium sorbate 64 of the preservatives 58 of the detoxifying and impurity eliminator 30;
Regulates production and release of sebum from the scalp 16, which is accomplished by the melatonin 36 of the anti-fungal and scalp metabolism regulator 26;
Detoxifies the scalp 16, which is accomplished by the imidazolidyny urea 56, the multi-functional polyethylene glycol 37, and the sodium bicarbonate 54;
Stabilizes the structure of substances integrated into it, and thus, prolongs their activity in a specific way, delivers target ingredients to target tissues, which is accomplished by the anti-fungal and scalp metabolism regulator 26;
Provides fungistatic and bacteriostatic properties to the scalp 16, which is accomplished by the zinc pyrithione 32 of the anti-fungal and scalp metabolism regulator 26;
Provides antifungal properties, which is accomplished by the climbazole 34 of the anti-fungal and scalp metabolism regulator 26;
Synchronizes circadian rhythms of the scalp 16, which is accomplished by the melatonin 36 of the anti-fungal and scalp metabolism regulator 26;
Eliminates pathogenic microorganism and exo- and endotoxins, which is accomplished by the white activated charcoal powder 66 of the pathogenic microorganism and exo- and endotoxins eliminator 60 of the detoxifying and impurity eliminator 30;
Binds to harmful fungi and fungal debris and byproducts of the scalp 16, which is accomplished by the white activated charcoal powder 66 of the pathogenic microorganism and exo- and endotoxins eliminator 60 of the detoxifying and impurity eliminator 30;
Removes harmful fungi and fungal debris and byproducts from the scalp 16, which is accomplished by the white activated charcoal powder 66 of the pathogenic microorganism and exo- and endotoxins eliminator 60 of the detoxifying and impurity eliminator 30;
Regulates toxicity and detoxifies the scalp 16, which is accomplished by the white activated charcoal powder 66 of the pathogenic microorganism and exo- and endotoxins eliminator 60 of the detoxifying and impurity eliminator 30;
Reduces toxicity and detoxifies the scalp 16, which is accomplished by the white activated charcoal powder 66 of the pathogenic microorganism and exo- and endotoxins eliminator 60 of the detoxifying and impurity eliminator 30;
Strengthens immune defense of the scalp 16, which is accomplished by the white activated charcoal powder 66 of the pathogenic microorganism and exo- and endotoxins eliminator 60 of the detoxifying and impurity eliminator 30;
Starts to work immediately after application to the scalp 16, which is accomplished by the white activated charcoal powder 66 of the pathogenic microorganism and exo- and endotoxins eliminator 60 of the detoxifying and impurity eliminator 30;
Protects the scalp 16 from harsh environmental influences, which is accomplished by the white activated charcoal powder 66 of the pathogenic microorganism and exo- and endotoxins eliminator 60 of the detoxifying and impurity eliminator 30;
Does not irritate the scalp 16, which is accomplished by the white activated charcoal powder 66 of the pathogenic microorganism and exo- and endotoxins eliminator 60 of the detoxifying and impurity eliminator 30;
Is completely eliminated from the scalp 16 by water, which is accomplished by the white activated charcoal powder 66 of the pathogenic microorganism and exo- and endotoxins eliminator 60 of the detoxifying and impurity eliminator 30; and
Does not interfere with hair colorants on the scalp 16, which is accomplished by the white activated charcoal powder 66 of the pathogenic microorganism and exo- and endotoxins eliminator 60 of the detoxifying and impurity eliminator 30,

### Impressions

It will be understood that each of the elements described above or two or more together may also find a useful application in other types of compositions differing from the types described above.

While the embodiments of the present invention have been illustrated and described as embodied in a topical composition for treating dandruff and seborrheic dermatitis of a scalp, by restoring architectonics to the scalp, by eliminating fungal infection from the scalp, by regulating metabolism of the scalp, by regulating production and release of sebum from the scalp, and by detoxifying the scalp, nevertheless, they are not limited to the details shown, since it will be understood that various omissions, modifications, substitutions, and changes in the forms and details of the embodiments of the present invention illustrated and their operation can be made by those skilled in the art without departing in any way from the spirit of the embodiments of the present invention.

Without further analysis, the foregoing will so fully reveal the gist of the embodiments of the present invention that others can by applying current knowledge readily adapt them for various applications without omitting features that from the standpoint of prior art fairly constitute characteristics of the generic or specific aspects of the embodiments of the present invention.

The invention claimed is:

1. A topical composition for treating dandruff and seborrheic dermatitis of a scalp, by restoring architectonics to the scalp, by eliminating fungal infection from the scalp, by regulating metabolism of the scalp, by regulating production and release of sebum from the scalp, and by detoxifying the scalp, said topical composition comprising:
a) an anti-fungal and scalp metabolism regulator;
b) a delivery system; and
c) a detoxifying and impurity eliminator;
wherein said detoxifying and impurity eliminator includes a pathogenic microorganism and exo- and endotoxins eliminator; and
wherein said pathogenic microorganism and exo- and endotoxins eliminator of said detoxifying and impurity eliminator includes white activated charcoal powder.

2. The topical composition of claim 1, wherein said anti-fungal and scalp metabolism regulator includes:
a) zinc pyrithione having fungistatic and bacteriostatic properties;
b) climbazole being a topical antifungal agent;
c) melatonin being a hormone involved in synchronizing circadian rhythms;
d) multi-functional polyethylene glycol; and
e) hydroxypropyl-ß-cyclodextrin (HPBC).

3. The topical composition of claim 2, wherein:
a) said zinc pyrithione of said anti-fungal and scalp metabolism regulator is in a concentration range of 0.05-5.7% wt/wt;
b) said climbazole of said anti-fungal and scalp metabolism regulator is in a concentration range of 1.0-6.0% wt/wt; and
c) said melatonin of said anti-fungal and scalp metabolism regulator is in a concentration range of 0.03-3.7% wt/wt.

4. The topical composition of claim 2, wherein:
a) said zinc pyrithione of said anti-fungal and scalp metabolism regulator is in a concentration range of 0.01-10% wt/wt;
b) said climbazole of said anti-fungal and scalp metabolism regulator is in a concentration range of 0.5-10% wt/wt; and
c) said melatonin of said anti-fungal and scalp metabolism regulator is in a concentration range of 0.01-5% wt/wt.

5. The topical composition of claim 2, wherein:
a) said zinc pyrithione of said anti-fungal and scalp metabolism regulator is in a concentration range of 0.05-10% wt/wt;
b) said climbazole of said anti-fungal and scalp metabolism regulator is in a concentration range of 0.75-10% wt/wt; and
c) said melatonin of said anti-fungal and scalp metabolism regulator is in a concentration range of 0.02-4% wt/wt.

6. The topical composition of claim 2, wherein said zinc pyrithione of said anti-fungal and scalp metabolism regulator is in a concentration range of 0.2-10% wt/wt.

7. The topical composition of claim 1, wherein said delivery system includes:
a) a first solution; and
b) a second solution.

8. The topical composition of claim 7, wherein said first solution of said delivery system includes:
a) dimethylsulfoxide (DMSO); and
b) hydroxypropyl-ß-cyclodextrin (HPBC).

9. The topical composition of claim 8, wherein:
a) said dimethylsulfoxide (DMSO) of said first solution of said delivery system is in a concentration range of 4.0-36.0% wt/wt; and
b) said hydroxypropyl-ß-cyclodextrin (HPBC) of said first solution of said delivery system is in a concentration range of 0.75-27.0% wt/wt.

10. The topical composition of claim 7, wherein said second solution of said delivery system includes:
a) distilled water;
b) sodium chloride;
c) potassium chloride;
d) calcium chloride;
e) sodium bicarbonate;
f) imidazolidinyl urea; and
g) multi-functional polyethylene glycol.

11. The topical composition of claim 10, wherein:
a) said distilled water of said second solution of said delivery system is in a concentration range of 8.0-46.0% wt/wt;
b) said potassium chloride of said second solution of said delivery system is in a concentration range of 0.7-2.8% wt/wt;
c) said calcium chloride of said second solution of said delivery system is in a concentration range of 0.03-5.2% wt/wt;
d) said sodium bicarbonate of said second solution of said delivery system is in a concentration range of 0.02-6.1% wt/wt; and
e) said imidazolidinyl urea of said second solution of said delivery system is in a concentration range of 0.1-4.5% wt/wt.

12. The topical composition of claim 10, wherein:
a) said distilled water of said second solution of said delivery system is in a concentration range of 8-90% wt/wt;
b) said potassium chloride of said second solution of said delivery system is in a concentration range of 0.01-5% wt/wt;
c) said calcium chloride of said second solution of said delivery system is in a concentration range of 0.01-5% wt/wt;
d) said sodium bicarbonate of said second solution of said delivery system is in a concentration range of 0.01-10% wt/wt; and
e) said imidazolidinyl urea of said second solution of said delivery system is in a concentration range of 0.01-10% wt/wt.

13. The topical composition of claim 1, wherein said detoxifying and impurity eliminator includes:
a) preservatives;
b) a pathogenic microorganism and exo- and endotoxins eliminator; and
c) multi-functional polyethylene glycol.

14. The topical composition of claim 13, wherein said preservatives of said detoxifying and impurity eliminator include:
a) sodium benzoate; and
b) potassium sorbate.

15. The topical composition of claim 14, wherein:
a) said sodium benzoate of said preservatives of said detoxifying and impurity eliminator is in a concentration of 0.500% wt/wt; and
b) said potassium sorbate of said preservatives of said detoxifying and impurity eliminator is in a concentration of 0.050% wt/wt.

16. The topical composition of claim 1, wherein said white activated charcoal powder of said pathogenic microorganism and exo- and endotoxins eliminator of said detoxifying and impurity eliminator:
a) is 10% of said topical composition; and
b) includes colloidal silicon dioxide;
wherein said colloidal silicon dioxide of said white activated charcoal powder of said pathogenic microorganism and exo- and endotoxins eliminator of said detoxifying and impurity eliminator is submicroscopic fumed silica prepared by vapor-phase hydrolysis of a silicon compound.

17. The topical composition of claim 1, wherein said white activated charcoal powder of said pathogenic microorganism and exo- and endotoxins eliminator of said detoxifying and impurity eliminator:
a) binds harmful fungi and fungal debris and byproducts of the scalp;
b) removes harmful fungi and fungal debris and byproducts from the scalp;
c) regulates and reduces toxicity and detoxifies the scalp;
d) strengthens immune defense of the scalp;
e) starts to work immediately after application to the scalp;
f) protects the scalp from harsh environmental influences;
g) does not irritate the scalp;
h) is completely eliminated from the scalp by water; and
i) does not interfere with hair colorants.

18. The topical composition of claim 1, wherein said topical composition:
a) restores architectonics to the scalp;
b) eliminates fungal infection from the scalp;
c) regulates metabolism of the scalp;
d) regulates production and release of sebum from the scalp;
e) detoxifies the scalp;
f) stabilizes structure of substances integrated into it, and thus, prolongs their activity in a specific way, and delivers target ingredients to target tissues,
g) provides fungistatic and bacteriostatic properties to the scalp;
h) provides antifungal properties;
i) synchronizes circadian rhythms of the scalp;
j) binds to harmful fungi and fungal debris and byproducts of the scalp;
k) removes harmful fungi and fungal debris and byproducts from the scalp;
l) regulates toxicity and detoxifies the scalp;
m) reduces toxicity and detoxifies the scalp;
n) strengthens immune defense of the scalp;
o) starts to work immediately after application to the scalp;
p) protects the scalp from harsh environmental influences;
q) does not irritate the scalp;
r) is completely eliminated from the scalp by water; and
s) does not interfere with hair colorants on the scalp.

19. The topical composition of claim 2, wherein:
a) said anti-fungal and scalp metabolism regulator stabilizes structure of substances integrated into it, and thus, prolongs their activity in a specific way, and delivers target ingredients to target tissues;
b) said zinc pyrithione of said anti-fungal and scalp metabolism regulator provides fungistatic and bacteriostatic properties to the scalp;
c) said climbazole of said anti-fungal and scalp metabolism regulator provides antifungal properties;
d) said melatonin of said anti-fungal and scalp metabolism regulator synchronizes circadian rhythms of the scalp;
e) said white activated charcoal powder of said pathogenic microorganism and exo- and endotoxins eliminator of said detoxifying and impurity eliminator:
i) eliminates pathogenic microorganism and exo- and endotoxins from the scalp;
ii) binds to harmful fungi and fungal debris and byproducts of the scalp;
iii) removes harmful fungi and fungal debris and byproducts from the scalp;
iv) regulates toxicity and detoxifies the scalp;
v) reduces toxicity of the scalp;
vi) strengthens immune defense of the scalp;
vii) starts to work immediately after application to the scalp;
viii) protects the scalp from harsh environmental influences;
ix) does not irritate the scalp;
x) is completely eliminated from the scalp by water; and
xi) does not interfere with hair colorants on the scalp.

20. The topical composition of claim 1, further comprising a fragrance.
